(19) United States
(12) Patent Application Publication
(10) Pub. No.: US 2026/0306645 A1
(43) Pub. Date: Oct. 8, 2026

(54) Modular platform for molecular conjugation

(71) Applicant: CamBrixBio Ltd, Ely (GB)

(72) Inventors: Maxim Rossmann, Ely (GB); Christine Hilcenko, Ely (GB); Richard Sainson, Ely (GB)

(21) Appl. No.: 19/637,072

(22) Filed: Apr. 2, 2026

(30) Foreign Application Priority Data
Apr. 7, 2025 (GB) 2505189.7

(51) Int. Cl. A61K 47/64; A61K 47/54; A61K 49/08; C07K 19/00; C12N 15/115

(52) U.S. Cl. CPC A61K 47/64; A61K 47/549; A61K 49/085; C12N 15/115; C07K 19/00; C12N 2310/16

(57) ABSTRACT

The present invention provides a modular platform for molecular conjugation, comprising a first module and a second module linked together, characterized in that: the first module comprises a peptide, a molecular staple, and a first L-nucleotide strand, and the second module comprises a second L-nucleotide strand, at least one linker, and at least one active molecule, wherein the second L-nucleotide strand is complementary to the first L-nucleotide strand, and wherein the peptide and the at least one active molecule are linked together through the first and the second L-nucleotide strands hybridising to form a double-stranded duplex.

### FIELD

The present invention relates to a modular platform for molecular conjugation, suitable in use in particular in the field of targeted drug delivery, diagnostics, molecular imaging, and theranostics.

### BACKGROUND

The conjugation of active molecules to peptides targeted against disease relevant proteins is a fundamental technique in the development of therapeutics, diagnostics, and molecular imaging agents. Peptides are valuable molecular carriers due to their biocompatibility, target specificity, and ability to modulate biological functions. However, effective conjugation strategies must balance stability, bioactivity, and precise molecular assembly.

Existing conjugation methods typically involve covalent attachment through chemical linkers, enzymatic modification, or non-covalent interactions. Antibody—drug conjugates (ADCs) consist of antibodies targeting specific antigens linked to cytotoxic payloads, enabling precise drug delivery. While these approaches can be effective, they often introduce challenges such as limited control over conjugation sites, reduced structural integrity of the peptide, and potential loss of biological activity, as well as reduced responsiveness to treatment. Additionally, conjugation can alter the pharmacokinetic profile of the peptide, affecting its stability, clearance rate, and immune recognition.

There remains a need for an improved and flexible conjugation strategy that ensures stability, specificity, and modularity while maintaining the functional integrity of both the peptide and the active molecule. An improved modular platform would enable precise molecular design for enhanced therapeutic, diagnostic, and imaging applications.

There are several known molecular conjugation platforms.

EP2911700B1 describes conjugates containing auristatins and a binding protein or peptide, and processes for making them. Also described are specific conjugates of drugs and a binding protein or peptide in which more than one copy of the drug is present.

CN113164621A describes a protein-drug conjugate comprising an antigen binding protein moiety and a drug conjugate moiety; the antigen binding protein portion comprises one or more antigen binding fragments, and at least two linking peptides directly or indirectly linked to the antigen binding fragments; the two linking peptides may each independently contain a first cysteine Cys1 and a second cysteine Cys 2; the drug conjugate moiety is covalently bound to the Cys 1. The application also relates to a preparation method of the protein-drug conjugate and application of the protein-drug conjugate in preventing or treating tumors or other diseases.

U.S. Pat. No. 9,849,191B2 describes a polymeric scaffold useful for conjugating with a protein based recognition-molecule (PBRM) to form a PBRM-polymer-drug conjugate. The scaffold includes one or more terminal maleimido groups. Also disclosed is a PBRM-polymer-drug conjugate prepared from the scaffold. Compositions comprising the conjugates, methods of their preparation, and methods of treating various disorders with the conjugates or their compositions are also described.

US20240216519A1 describes peptide conjugated drugs based on MuV SH Protein, their use as therapeutic agents and their use to provide delivery to and/or transfer across a membrane of the drug. It provides use of the peptide conjugated drug for delivery of the drug to and/or across a membrane harbouring G protein-coupled receptor 125 (GPR125), such as the choroid plexus.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art or form part of the common general knowledge in the art.

### SUMMARY

It is an object of the present invention to provide a modular platform for molecular conjugation which goes some way to overcoming the disadvantages of the solutions described in the abovementioned documents or which at least provides the public or industry with a useful choice.

The present invention further aims to provide a modular platform for the flexible and precise molecular assembly of one or more payloads onto a stapled peptide. Further, the aim is to provide a platform that will enable the conjugation of identical or distinct payloads, allowing multiple payloads to be delivered to the same cell simultaneously. Alternatively, when a single type of payload is attached, the aim of the system is to enhance intracellular concentration of that payload.

Additionally, the present invention seeks to provide a modular platform composed of two interchangeable modules that can be mixed and matched to accommodate different peptides and active molecules while maintaining compatibility through complementary L-nucleotide strand hybridization.

The present disclosure also seeks to provide a method for preparing the modular platform.

The term “comprising” as used in this specification and indicative independent claims means “consisting at least in part of”. When interpreting each statement in this specification and indicative independent claims that include the term “comprising”, features other than that or those prefaced by the term may also be present. Related terms such as “comprise” and “comprises” are to be interpreted in the same manner.

As used herein the term “and/or” means “and” or “or”, or both.

As used herein “(s)” following a noun means the plural and/or singular forms of the noun.

Accordingly, in a first aspect, the present invention may broadly be said to provide a modular platform for molecular conjugation, comprising a first module and a second module linked together, characterized in that: the first module comprises a peptide, a molecular staple, and a first L-nucleotide strand, and the second module comprises a second L-nucleotide strand, at least one linker, and at least one active molecule, wherein the second L-nucleotide strand is complementary to the first L-nucleotide strand, and wherein the peptide and the at least one active molecule are linked together through the first and the second L-nucleotide strands hybridising to form a double-stranded duplex.

In an embodiment, the modular platform for molecular conjugation comprises two or more copies of the second module.

In an embodiment, the second module comprises two active molecules.

In an embodiment, the second module comprises two different active molecules.

In an embodiment, the active molecule is one of: a cytotoxic agent, an immunostimulatory agent, an immunosuppressive agent, a radiosensitiser.

In an embodiment, the active molecule is one of: a peptide, a protein, a nucleic acid, a nanobody.

In an embodiment, wherein the active molecule is an aptamer.

In an embodiment, the active molecule is one of: a Positron Emission Tomography (PET) tracer, a fluorescent dye, a contrast agent, a paramagnetic molecule, an ultrasound sensitiser, a radioactive isotope, an alpha-emitter, a beta emitter, a gold-based agent.

In an embodiment, the contrast agent is gadolinium-based, iron-based, or manganese-based.

In an embodiment, wherein the staple contains at least one functional group enabling formation of a covalent bond with the first L-nucleotide strand.

In an embodiment, the staple contains at least one of: a bromomethyl group, an azide group.

The modular platform of claims 1-11, wherein the staple contains at least one of: 1,3,5-tris(bromomethyl)benzene (1,3,5-TBMB), 1,2,3-tris(bromomethyl)benzene (1,2,3-TBMB), 1,2,3,5-tetra(bromomethyl)benzene (1,2,3,5-TBMB).

In an embodiment, the hybridisation of the first and the second L-nucleotide strands is reversible, allowing dynamic assembly and disassembly of the conjugation system.

In an embodiment, the length of the first L-nucleotide strands is between 15 and 80 nucleotides.

In an embodiment, the length of the second L-nucleotide strands is between 15 and 40 nucleotides.

In an embodiment, one or both L-nucleotide strands is L-RNA.

In an embodiment, the first and/or the second L-nucleotide strands are chemically modified to contain at least one thiol group.

In an embodiment, the thiol group is located at the 5′ end of the first and/or the second L-nucleotide strand.

In an embodiment, the thiol group is located at the 3′ end of the first and/or the second L-nucleotide strand.

In an embodiment, the first and/or the second L-nucleotide strands are chemically modified to contain at least one amine group.

In an embodiment, the amine group is located at the 5′ and/or 3′ end of the first and/or the second L-nucleotide strand.

in a second aspect, the present invention may broadly be said to provide a method for preparing the modular platform for the conjugation of a peptide with an active molecule, the method comprising: conjugation of a staple of the peptide to a first L-nucleotide strand, stapling of a Cysteine-containing peptide, conjugation of the active molecule to a second L-nucleotide strand, and hybridisation of the first and the second L-nucleotide strands and formation of a double-stranded duplex.

In an embodiment, after thermal dissociation of the double-stranded duplex, the second module may be replaced by an alternative second module comprising a different active molecule.

In an embodiment, the hybridisation between the first and second L-nucleotide strands is urea, and/or acetonitrile sensitive, and dissociation of the duplex can be induced by incubation of the system in urea, formamide, and/or acetonitrile solution.

In an embodiment, after chemical dissociation, the second module may be replaced by an alternative second module comprising a different active molecule and a second L-nucleotide strand complementary to the first L-nucleotide strand.

Additional aspects, advantages, features and objects of the present disclosure would be made apparent from the drawings and the detailed description of the illustrative embodiments construed in conjunction with the appended claims that follow.

With respect to the above description then, it is to be realised that the optimum dimensional relationships for the parts of the invention, to include variations in size, form, function and manner of operation, assembly and use, are deemed readily apparent and obvious to one skilled in the art, and all equivalent relationships to those illustrated in the drawings and described in the specification are intended to be encompassed by the present invention.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### BRIEF DESCRIPTION OF THE DRAWINGS

FIG. 1A shows the first module of the modular platform for molecular conjugation, comprising a peptide, a molecular staple, and a first L-nucleotide strand, in accordance with an embodiment of the present disclosure.

FIG. 1B shows the second module of the modular platform for molecular conjugation, comprising a second L-nucleotide strand, a linker, and an active molecule, in accordance with an embodiment of the present disclosure.

FIG. 1C shows the first and the second modules after hybridization of the first and the second L-nucleotide strands forming a conjugated molecule, in accordance with an embodiment of the present disclosure.

FIG. 1D shows one copy of the first module and two copies of the second module after hybridization of the first and the second L-nucleotide strands forming a conjugated molecule, in accordance with an embodiment of the present disclosure.

FIG. 2A shows the second module of the modular platform for molecular conjugation, comprising a second L-nucleotide strand, two copies of a linker, and two copies of an active molecule, attached at the two ends of the second L-nucleotide strand, in accordance with an embodiment of the present disclosure.

FIG. 2B shows the first and the second modules after hybridization of the first and the second L-nucleotide strands, where the second module comprises two copies of an active molecule, forming a conjugated molecule, in accordance with an embodiment of the present disclosure.

FIG. 3A shows the second module of the modular platform for molecular conjugation, comprising two copies of a second L-nucleotide strand, two copies of a linker, and two copies of an active molecule, where the two copies of the L-nucleotide strands are attached together, the linkers are attached at each end of the strands, and the two copies of the active molecule are attached to the linkers, in accordance with an embodiment of the present disclosure.

FIG. 3B shows one copy of the first module and two copies of the second module after hybridization, where the two copies of the second module are not identical, and where the first copy of the second module hybridises to the first module, and the second copy of the second module hybridises to the first copy of the second module, in accordance with an embodiment of the present disclosure.

FIG. 4 shows binding of the peptide to the molecular staple with the first L-nucleotide strand attached, in accordance with an embodiment of the present disclosure.

### DETAILED DESCRIPTION

Non-limiting embodiments of the invention, and variations thereof, will now be described in detail.

### General

The present invention provides a novel modular platform for linking active molecules to peptides via hybridisation of complementary L-nucleotide strands. This system offers a stable, modular, and highly specific approach to peptide conjugation, addressing several limitations of existing chemical and enzymatic conjugation methods. Namely, this system offers increased in vivo stability, and reduced in vivo immunogenicity.

The present invention provides a novel modular platform designed for flexible and precise molecular assembly, comprising two interchangeable modules that can be mixed and matched while maintaining compatibility through complementary L-nucleotide strand hybridization.

As disclosed in the non-limiting example, the first module comprises a peptide targeted against disease relevant proteins, a molecular staple, and the first L-nucleotide strand, where the peptide serves as the targeting or functional component, the staple stabilizes its structure, and the first L-nucleotide strand enables hybridization to one or more therapeutic agent. The second module comprises a second L-nucleotide strand, at least one linker, and at least one active molecule, with the second L-nucleotide strand hybridizing specifically with the first to form a stable duplex, the linker facilitating covalent attachment, and the active molecule providing therapeutic, diagnostic, or imaging functionality. By ensuring complementarity between the first and second L-nucleotide strands which allows hybridization, different peptides and active molecules can be interchanged without altering the core conjugation mechanism, making the system highly adaptable for applications in targeted drug delivery, molecular imaging, and theranostics.

In an embodiment of the invention, the first module hybridises to two or more copies of the second module. In this embodiment, the second L-nucleotide strands of the two copies of the second module are complementary to the first L-nucleotide strand of the first module and hybridise to the first L-nucleotide strand. The two copies of the second module can be identical or can be different. The sequences of the second L-nucleotide strands of the two copies of the second module can be identical or different.

In an embodiment of the invention, the second module comprises a second L-nucleotide strand, two copies of a linker, and two copies of an active molecule. The first copy of the linker is attached at the 5′ end of the second L-nucleotide strand, and the first copy of the active molecule is attached to the first copy of the linker. The second copy of the linker is attached at the 3′ end of the second L-nucleotide strand, and the second copy of the active molecule is attached to the second copy of the linker. Complementarity between the first L-nucleotide strand and the second L-nucleotide strand allows hybridisation.

In the embodiment of the invention, the second module comprises two copies of a second L-nucleotide strand (identical or non-identical), two copies of a linker, and two copies of an active molecule. The 5′ end of the first copy of the second L-nucleotide strand is attached to the 3′ end of the second copy of the second L-nucleotide strand. The first copy of the linker is attached to the 5′ end of the second copy of the second L-nucleotide strand. The first copy of the active molecule is attached to the first copy of the linker. The second copy of the linker is attached to the 3′ end of the first copy of the second L-nucleotide strand. The second copy of the active molecule is attached to the second copy of the linker.

In this embodiment, one copy of the first module and two copies of the second module hybridise. The two copies of the second module are not identical. The first copy of the second module hybridises to the first module, and the second copy of the second module hybridises to the first copy of the second module.

The modular platform for molecular conjugation can be characterised using a peptide to drug ratio (PDR). The PDR is defined by the number of active molecules present compared to the number of peptides.

In an embodiment, the first module hybridises with one copy of the second module, wherein the second module comprises one active molecule. This results in a PDR of 1.

In an embodiment, the first module hybridises with one copy of the second module, wherein the second module comprises two active molecules. The two active molecules can be the same or different. This results in a PDR of 2.

In an embodiment, the first module hybridises with two copies of the second module. The two copies of the second module are identical. This results in a PDR of 2.

In an embodiment, the first module hybridises with two copies of the second module. The two copies of the second module are not identical, each comprising a different active molecule. This results in a PDR of 2. The peptide comprises at least two reactive sites that allow it to bind to the first L-nucleotide strand. The first L-nucleotide strand, in turn, is conjugated with a staple that contains complementary reactive sites to those on the peptide. The second L-nucleotide strand is complementary to the first L-nucleotide strand. The second L-nucleotide strand carries the active molecule ensuring stable linkage. When the two L-nucleotide strands hybridize, they create a stable molecular bridge that connects the active molecule to the peptide in a controlled manner.

The L-nucleotide strands do not hybridize with natural D-DNA or D-RNA, minimizing the likelihood of off-target interactions. The L-nucleotides can be PEGylated or conjugated to carriers to adjust clearance rates.

This modular approach enables the attachment of a wide range of active molecules (as monomer, polymer or combination of monomers), including therapeutic agents such as, but not limited to, cytotoxic drugs, immunostimulatory drugs, immunosuppressive drugs, radiotherapeutics, or radiosensitizers, as well as diagnostic and imaging probes such as fluorescent dyes, PET tracers, and MRI contrast agents. By utilizing complementary hybridization, the system allows for tuneable conjugation, providing flexibility in drug design and molecular imaging applications.

By addressing the limitations of traditional conjugation methods, this invention provides a robust and adaptable platform for peptide-based therapeutics and diagnostics development. It represents a significant advancement over existing oligonucleotide-based conjugation techniques by combining the stability of L-nucleotides, the versatility of stapled peptides, and the specificity of molecular hybridisation.

### The Peptide

The peptide serves as the targeting and functional component of the modular platform, enabling selective interaction with biological targets for therapeutic or diagnostic applications. It is designed to retain high specificity and selectivity and binding affinity while allowing for stable conjugation to the molecular scaffold. The peptide is selected or engineered based on its ability to recognize and bind to a disease-relevant target, such as a protein involved in autoimmune diseases, cancer progression, a marker upregulated in cancer, or other pathological processes.

To facilitate conjugation, the peptide includes reactive sites that allow it to bind to the first L-nucleotide strand. The peptide will contain a pre-selected number of cysteine residues or other functional groups that can form stable linkages with the adaptor strand. The positioning of these reactive sites is carefully optimized to ensure that conjugation does not interfere with the peptide's binding ability or biological activity.

The peptide can be of any size. The peptide is typically between 8-12 amino acids in size, making it small enough for efficient tissue penetration while maintaining sufficient structural integrity for stable interactions with its target. The peptide contains at least two Cysteine residues (e.g., Met-Asp-xxx-xxx-xxx-Cys-xxx-xxx-xxx-Cys-xxx-xxx-xxx, where xxx represents amino acids in the peptide sequence). The peptide is designed to achieve high affinity, optimal stability and solubility under physiological conditions, reducing the likelihood of aggregation or degradation. Additionally, its sequence and structure are optimized to minimize immunogenicity, ensuring compatibility with therapeutic applications.

A key aspect of the peptide's development involves high-throughput screening and selection to identify candidates with strong binding affinity and specificity. This is achieved by generating a diverse library of peptide sequences containing strategically positioned reactive sites for conjugation. These sequences are screened against immobilized target molecules, and the most effective binders are selected for further optimization. Machine learning/Artificial intelligence and molecular modelling techniques may also be employed to refine peptide sequence and structure, improving binding affinity while enhancing stability and manufacturability.

Once conjugated to the first L-nucleotide chain, the peptide retains its target-binding properties while benefiting from the stability and modularity provided by the L-nucleotide scaffold. This allows for precise control over drug delivery, enabling the peptide to function as a targeting mechanism that directs therapeutic or diagnostic payloads to specific cells or tissues. The conjugation process ensures that the active molecule remains securely attached without disrupting the natural function of the peptide, making it a highly effective component for precision medicine applications. The conjugation strategy provides stability both on the shelf—ensuring long-term storage without degradation—and in vivo, maintaining structural integrity and bioactivity after administration.

The modular nature of the platform allows for different peptides to be incorporated based on the intended application. For therapeutic use, the peptide may be selected to inhibit disease-related pathways, modulate immune responses, or deliver cytotoxic agents directly to cancer cells. In an embodiment, the peptide can be taken up by target cells for intracellular delivery of the payloads. In diagnostic applications, the peptide may be conjugated to imaging agents to enhance the detection of specific biomarkers, monitor disease progression. Regardless of the application, the peptide remains a critical element in ensuring specificity, stability, and functional effectiveness within the modular platform.

### The Staple

The staple connects the peptide and the first L-nucleotide chain.

In an embodiment, the staple contains functional groups enabling the formation of a covalent bond with the first L-nucleotide strand.

In an embodiment, the staple contains at least one of: a bromomethyl group, an azide group.

In an embodiment, the staple contains at least one of: 1,3,5-tris(bromomethyl)benzene (1,3,5-TBMB), 1,2,3-tris(bromomethyl)benzene (1,2,3-TBMB), 1,2,3,5-tetra(bromomethyl)benzene (1,2,3,5-TBMB).

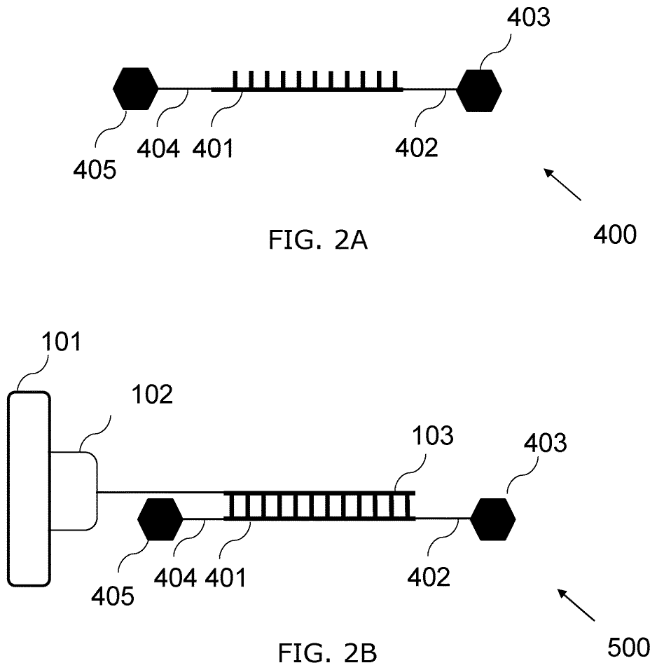

In an embodiment, the staple contains 1,3,5-tris(bromomethyl)benzene (1,3,5-TBMB). As seen in FIG. 2, the staple is conjugated to the thiol groups of the Cysteins present in the peptide.

Stapled peptides are a class of therapeutic molecules that offer distinct advantages over monoclonal antibodies (mAbs) and traditional small molecules. They have a smaller size than monoclonal antibodies, allowing for better tumour penetration and diffusion into the tumour microenvironment and targeting of intracellular protein-protein interactions. Stapled peptides have improved pharmacokinetics, higher affinity to the target, a longer half-life and better bioavailability than linear peptides. Stapled peptides have enhanced protease resistance, making them more stable in serum and extracellular environments.

Stapled peptides have a theranostic potential, as their size and pharmacokinetic profile make them ideal for conjugation with imaging agents (PET, MRI) and therapeutic payloads, enabling real-time tracking of drug distribution and efficacy. Stapled peptides have high scalability and manufacturability, with lower production costs compared to monoclonal antibodies.

### The L-Nucleotide Strands

The L-nucleotide strands form the molecular scaffold that enables the stable and precise conjugation of the peptide to the active molecule. These strands play a critical role in ensuring controlled molecular assembly by utilizing sequence-specific hybridization, which provides a highly modular and adaptable platform for conjugation. Unlike natural DNA and RNA, L-nucleotide strands are composed of L-DNA or L-RNA, which have the same chemical composition as their natural counterparts but feature a mirror-image stereochemistry. This fundamental difference makes L-nucleotides highly resistant to enzymatic degradation, ensuring superior stability in physiological environments. They also exhibit lower immunogenicity.

The use of L-nucleotides offers several advantages over traditional conjugation strategies. Natural DNA and RNA are susceptible to rapid degradation by nucleases, which limits their stability and lifespan in biological environments. In contrast, L-nucleotides are not recognized by these enzymes, resulting in extended half-life and enhanced durability in vivo. This ensures that the modular platform remains intact for prolonged periods, improving the pharmacokinetics of the peptide-active molecule complex. L-RNA single strand is more inert in the body than a L-DNA strand and is preferred over L-DNA.

Additionally, L-nucleotide strands can be chemically modified to facilitate covalent attachment of biomolecules. Functional groups such as thiol (—SH) or amine (—NH₂) moieties can be introduced at specific positions, enabling conjugation via well-established chemical reactions, including SN2 substitution or Schiff-base formation. This flexibility allows for the stable attachment of a variety of payloads, including therapeutic agents, diagnostic markers, and imaging probes.

Another key advantage of L-nucleotide-based conjugation is its reversibility. Unlike traditional covalent linkages that permanently modify the peptide, the hybridization between the two L-nucleotide strands is non-covalent and sequence-dependent. This enables dynamic control over molecular assembly, allowing for tuneable drug release. The ability to control the stability and dissociation of the complex offers significant benefits for applications requiring controlled drug delivery, real-time imaging, or sequential therapeutic interventions.

The sequence and length of the L-nucleotide strands are optimized to minimize off-target interactions while ensuring strong hybridization specificity. Typically, the strands are between 15 to 40 nucleotides in length, which is sufficient to maintain stability while preventing unintended binding to natural nucleic acids present in biological systems. To further enhance specificity, multiple sequence variants may be designed and screened to identify those with the highest hybridization efficiency and stability and minimal risk of non-specific interactions with host proteins or cells.

The L-nucleotide strand has a GC content of 40-60% to ensure structural integrity at 37° C. while avoiding excessive stability that could impede controlled interactions. The melting temperature exceeds 50° C., more specifically 60° C., to maintain L-nucleotide strand duplex stability under physiological conditions. To optimize hybridization efficiency, the sequence is designed to minimize secondary structures such as hairpins or self-dimers, which could interfere with strand pairing. The duplex is biologically inert, meaning it is not recognized by host proteins, a property that can be experimentally validated using an approach such as the Retrogenix platform. Computational tools such as NUPACK or RNAfold can assist in sequence optimization. Experimental validation is essential to confirm optimal hybridization and biological stability.

The modularity of the L-nucleotide scaffold enables broad adaptability across multiple biomedical applications. Different active molecules can be attached to the second L-nucleotide strand without altering the fundamental conjugation strategy, allowing for rapid customization based on therapeutic or diagnostic needs. This makes the system particularly advantageous for precision medicine applications, where targeted delivery and controlled release are critical.

The modular platform employs two distinct L-nucleotide strands, each serving a unique function. The first L-nucleotide strand acts as an adaptor, linking the peptide to the second L-nucleotide strand. The second L-nucleotide strand is responsible for carrying the active molecule and hybridizing with the first strand to complete the assembly. By hybridizing in a sequence-specific manner, the two L-nucleotide strands create a stable duplex that secures the active molecule in proximity to the peptide without requiring direct covalent modification of the peptide itself.

### The First L-Nucleotide Strand

The first L-nucleotide strand serves as an adaptor, linking the peptide to the second L-nucleotide strand carrying the active molecule. It facilitates precise molecular assembly while maintaining flexibility for different therapeutic and diagnostic applications.

This strand is a single-stranded L-DNA or L-RNA sequence, typically 15 to 80 nucleotides long, designed to hybridize specifically with the second L-nucleotide strand to form a stable duplex. The length is largely dependent on the desired PDR and number of second module copies bound by the first L-nucleotide strand. Due to its mirror-image stereochemistry, it is highly resistant to enzymatic degradation, ensuring superior stability in physiological environments.

The first L-nucleotide strand is linked to the staple. Sequence-specific hybridization with the second L-nucleotide strand ensures controlled assembly, making the system more adaptable than traditional covalent conjugation methods.

The first L-nucleotide strand can bind at least one second L-nucleotide strand of the second module. In an embodiment, the first L-nucleotide strand can bind two or more second L-nucleotide strands, which results in the binding of two or more copies of the second module. The two or more copies of the second module can be identical or different.

When the number of second modules and so second L-nucleotide strands is higher than one, the first L-nucleotide strand sequences which are complimentary to the second L-nucleotide strands are linked together by a spacer. The length of the spacer is typically 2-10 nucleotides long.

### The Second L-Nucleotide Strand

The second L-nucleotide strand enables the attachment of at least one linker (cleavable or non-cleavable) and at least one active molecule while hybridizing specifically with the first L-nucleotide strand, ensuring a stable and controlled conjugation process. It plays a crucial role in securing the active molecule in proximity and in a stable manner to the peptide while maintaining modularity and adaptability for different therapeutic and diagnostic applications.

This strand consists of single-stranded L-DNA or L-RNA, typically 15 to 40 nucleotides long, optimized for stability and specificity. Its L-nucleotide composition prevents enzymatic degradation, ensuring prolonged durability in biological environments. The sequence is designed to hybridize with the first strand with high specificity, minimizing off-target interactions and maintaining structural integrity under physiological conditions.

To enable active molecule attachment, the second L-nucleotide strand is chemically modified, typically at the 5′ end, with a reactive functional group such as thiol (—SH) or maleimide. This allows for stable covalent conjugation of a wide range of payloads, including therapeutic agents, diagnostic markers, and imaging probes. The linker chemistry can be cleavable or non-cleavable, depending on whether controlled release of the active molecule is required.

Hybridization between the first and second L-nucleotide strands ensures precise positioning of the active molecule relative to the peptide, improving targeting efficiency and therapeutic efficacy. This modular approach allows different payloads to be incorporated without altering the core modular platform, making it highly adaptable for targeted drug delivery, molecular imaging, and precision medicine applications.

In an embodiment, one linker is attached to the second L-nucleotide strand, at either the 3′ or the 5′ end, wherein the linker is attached to an active molecule.

In an embodiment, two of the same linker are attached to the second L-nucleotide strand, one at the 3′ end and one at the 5′ end. There is an active molecule attached to each of the linkers, making it two active molecules in total. The active molecules can be the same compound or two different compounds.

In an embodiment, two different linkers are attached to the second L-nucleotide strand, one at the 3′ end and one at the 5′ end. There is an active molecule attached to each of the linkers, making it two active molecules in total. The active molecules can be the same compound or two different compounds.

### The Linker

The linker attaches the second L-nucleotide strand to the active molecule.

In an embodiment, the linker is a sulphide bridge, and the active molecule is attached to the second L-nucleotide strand using a thiol crosslinking reaction. In an embodiment, the linker includes a cleavable site. In another embodiment, the linker is non-cleavable.

The linker comprises a functional group enabling the formation of a bond with the active molecule.

In a non-limiting example, the linker can be one of: hydrazone, disulphide, Valine-Citrulline (Val-Cit), Glycine-Phenylalanine-Leucine-Glycine (Gly-Phe-Leu-Gly), thioether (e.g., SMCC), maleimidocaproyl (MC), Glycine-Glycine-Phenylalanine-Glycine (GGFG).

### The Active Molecule

The active molecule serves as the functional component of the modular platform, providing therapeutic, diagnostic, or imaging capabilities. It is covalently attached to the second L-nucleotide strand, which hybridizes with the first L-nucleotide strand, ultimately linking it to the peptide. This structured assembly ensures precise positioning of the active molecule while preserving its biological activity and improving its stability in physiological environments.

A wide range of active molecules can be incorporated into the system, depending on the intended application. The modular platform may provide one active molecule attached to the second L-nucleotide strand or two (same or different) active molecules attached to the second L-nucleotide strand. If two second L-nucleotide strands are hybridized with the first L-nucleotide strand, the platform may provide up to four same or different active molecules. It is of particular importance in therapies.

In a non-limiting example, the active molecule can be one of: a cytotoxic agent, an immunostimulatory agent, an immunosuppressive agent, a radiosensitiser, an alpha emitter, a beta emitter, an auger electron emitter, a positron emitter, a brachytherapy emitter.

For therapeutic applications, the system can be conjugated with small-molecule drugs, peptides, proteins, or biologics. Examples include cytotoxic agents such as doxorubicin, paclitaxel, or gemcitabine for targeted cancer therapies, immunomodulatory compounds like checkpoint inhibitors (e.g., anti-PD-1 or anti-CTLA-4 antibodies) or cytokines (e.g., interleukins or interferons) for autoimmune and inflammatory diseases, and enzyme inhibitors such as kinase inhibitors (e.g., imatinib, gefitinib) or protease inhibitors for metabolic and infectious diseases. Additionally, gene therapy agents, including antisense oligonucleotides, siRNAs, or CRISPR/Cas components, can be incorporated for genetic modulation.

For diagnostic applications, the system can be conjugated with fluorescent dyes such as Alexa Fluor, FITC, or cyanine dyes for optical imaging, radioisotopes like technetium-99m, zirconium-89, or fluorine-18 for PET and SPECT imaging, and paramagnetic contrast agents such as gadolinium-based compounds for MRI. These payloads enable precise tracking, visualization, and quantification of molecular targets in real-time, improving diagnostic sensitivity and specificity.

For theranostic applications, the system can combine therapeutic and diagnostic functionalities in a single conjugate. Examples include radiopharmaceuticals like lutetium-177 and actinium-225, which provide both imaging and therapeutic capabilities for cancer treatment, as well as photodynamic therapy agents such as verteporfin and IRDye 700DX, which can be activated by light to generate localized cytotoxic effects while allowing for fluorescence-based imaging.

The active molecule is covalently linked to the second L-nucleotide strand via a reactive functional group, typically positioned at the 5′ end. Common attachment chemistries include thiol (—SH), maleimide, azide-alkyne click chemistry, or amide bond formation, ensuring stable and efficient conjugation. The linker can be cleavable or non-cleavable, depending on the desired release and bystander profile. Cleavable linkers, such as enzyme-sensitive linkers, allow for controlled release and local diffusion of the active molecule at the target site, which is particularly useful in drug delivery applications requiring precise activation. Non-cleavable linkers, such as stable thioether bonds, ensure that the active molecule remains bound throughout its functional lifespan, benefiting applications requiring prolonged retention, as well as reducing diffusion of highly toxic drugs in the periphery and at the site of activity.

The modularity of this platform allows for easy substitution of different active molecules without altering the core peptide or L-nucleotide scaffold. This adaptability enhances the platform's versatility in drug development, targeted therapies, and precision diagnostics. By maintaining a stable and controlled molecular assembly, the system ensures that the active molecule retains its intended function while benefiting from improved pharmacokinetics, reduced off-target effects, and enhanced therapeutic or diagnostic precision

The modular platform as described in the present invention may be obtained via any method suitable for creation of this modular platform. It may be also realized by the method described in this application. Both the method and module described below should be considered as a non-limiting example of the realization of the present invention.

In a non-limiting embodiment, a TBMB-stapled peptide can be conjugated to a gold nanoparticle through L-DNA strand hybridization.

### Synthesis and Modification of L-DNA Oligomers

Two complementary L-DNA strands, each 18 base pairs in length with a GC content of 50%, were synthesized to provide a stable hybridization scaffold. The sequences were designed to ensure thermal stability at physiological conditions, with a melting temperature above 50° C. Each strand was modified at the 5′-end with a thiol (—SH) group to facilitate covalent conjugation to either the peptide or a gold nanoparticle.

The first L-DNA strand was functionalized for peptide conjugation, while the second strand was designed for gold nanoparticle attachment. These modifications enabled a controlled assembly process in which the L-DNA duplex served as a molecular bridge linking the peptide to the nanoparticle.

### Conjugation of the First L-DNA Strand to a Stapled Peptide

The first L-DNA strand was covalently linked to a peptide through a TBMB-based crosslinking reaction. Initially, 1,2,3,5-tetra(bromomethyl)benzene (1,2,3,5-TBMB) was activated in an anhydrous solvent, such as DMF or DMSO, under mild basic conditions. The 5′-thiolated L-DNA strand was introduced into the reaction, allowing for SN2 substitution at the bromomethyl positions of TBMB, resulting in a stable covalent attachment of the L-DNA strand at the 5-position of the TBMB core.

A cysteine-containing peptide was then conjugated to the TBMB-functionalized L-DNA strand. The peptide, containing three cysteine residues spaced within its sequence, was dissolved in a mild basic buffer and incubated with the TBMB-L-DNA conjugate. The bromo groups of TBMB reacted with the thiol groups of the cysteine residues, forming stable covalent bonds that crosslinked the peptide. This stapling process stabilized the peptide's conformation and ensured its attachment to the L-DNA scaffold. The final peptide-L-DNA conjugate was purified using high-performance liquid chromatography (HPLC) or gel filtration chromatography.

### Conjugation of the Second L-DNA Strand to Gold Nanoparticles

Gold nanoparticles (AuNPs) were synthesized via citrate reduction of HAuCl₄ or obtained commercially in the 5-20 nm size range. The second L-DNA strand, functionalized with a 5′-thiol (—SH) group, was conjugated to the nanoparticles through Au—S bonding. This was achieved by incubating the thiolated L-DNA strand with the gold nanoparticles under slightly acidic conditions (pH ~6.5-7), promoting strong covalent interactions between the gold surface and the thiol groups. Excess unbound L-DNA was removed by centrifugation and washing steps in PBS buffer. The successful conjugation of L-DNA to the nanoparticles was confirmed using UV-Vis spectroscopy, which showed a shift in the plasmon resonance peak, and dynamic light scattering (DLS) measurements to assess particle size distribution.

### Formation of the L-DNA Hybridized Bridge

To assemble the final conjugate, equimolar amounts of the peptide-functionalized L-DNA strand and the gold nanoparticle-functionalized L-DNA strand were mixed in a hybridization buffer containing Tris, NaCl, and MgCl₂ at physiological pH. The solution was heated to 60° C. to disrupt any secondary structures and then gradually cooled to room temperature to allow controlled duplex formation. Successful hybridization of the two L-DNA strands was confirmed using UV-Vis absorbance melting curve analysis and gel electrophoresis, which demonstrated the formation of a stable L-DNA duplex.

### Final Product and Applications

The final gold nanoparticle-peptide hybrid was characterized using transmission electron microscopy (TEM) and surface plasmon resonance (SPR) measurements. This conjugation strategy, utilizing L-DNA as a hybridization scaffold, provides a highly stable and modular approach for nanoparticle-based targeted drug delivery, molecular imaging, and therapeutic applications. The stapled peptide enhances stability and bioactivity, while the gold nanoparticles enable multimodal imaging and therapeutic effects, including photothermal therapy, drug delivery, and catalytic applications. This system demonstrates the versatility of L-DNA-mediated conjugation, allowing for precise molecular assembly in advanced biomedical applications.

### DETAILED DESCRIPTION OF THE DRAWINGS

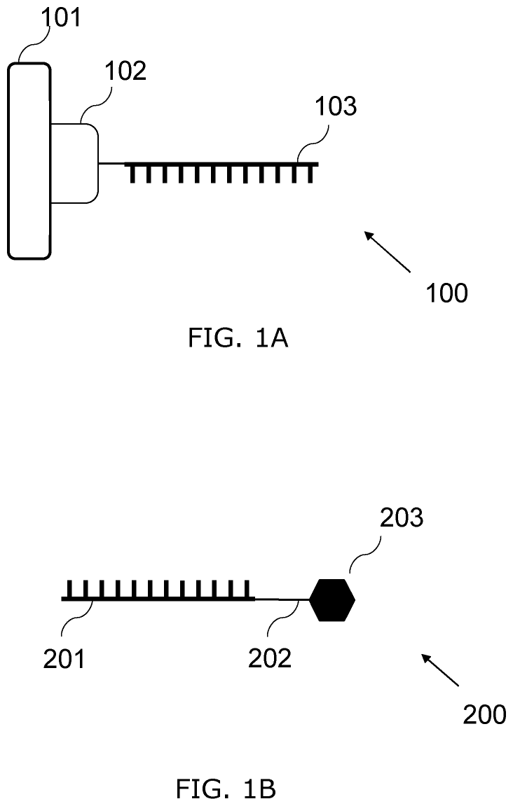

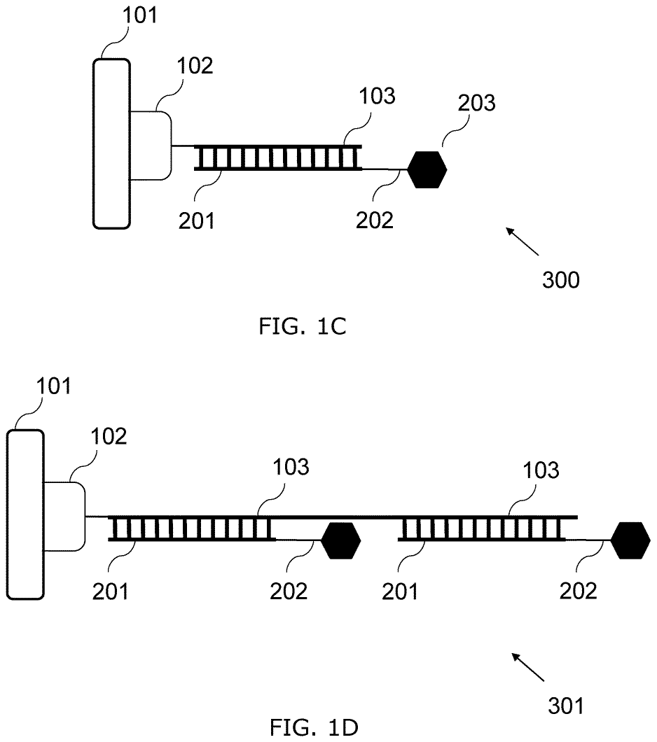

As disclosed in the non-limiting example (FIG. 1) the first module 100 comprises a peptide 101, a molecular staple 102, and the first L-nucleotide strand 103, where the peptide 101 serves as the targeting or functional component, the staple 102 stabilizes its structure, and the first L-nucleotide strand 103 enables hybridization. The second module 200 comprises a second L-nucleotide strand 201, a linker 202, and an active molecule 203, with the second L-nucleotide strand 201 hybridizing specifically with the first to form a stable duplex, the linker 202 facilitating covalent attachment, and the active molecule 203 providing therapeutic, diagnostic, or imaging functionality. By ensuring complementarity between the first and second L-nucleotide strands which allows hybridization 300, different peptides 101 and active molecules 203 can be interchanged without altering the core conjugation mechanism, making the system highly adaptable for applications in targeted drug delivery, molecular imaging, and theranostics.

The peptide 101 comprises at least two reactive sites that allow it to bind to the first L-nucleotide strand via the staple 103. The first L-nucleotide strand 103, in turn, is conjugated with a staple 102 that contains complementary reactive sites to those on the peptide 101. The second L-nucleotide strand 201 is complementary to the first L-nucleotide strand 103. The second L-nucleotide strand 201 carries the active molecule 203 through covalent attachment, ensuring stable linkage. When the two L-nucleotide strands hybridize, they create a stable molecular bridge that connects the active molecule 203 to the peptide 101 in a controlled manner.

In an embodiment of the invention 301, the first module 100 hybridises to two copies of the second module 200.

FIG. 2 shows an embodiment of the invention, where the second module 400 comprises a second L-nucleotide strand 401, two copies of a linker 402, 404, and two copies of an active molecule 403, 405. The first copy of the linker 402 is attached at the 5′ end of the second L-nucleotide strand 401, and the first copy of the active molecule 403 is attached to the first copy of the linker 402. The second copy of the linker 404 is attached at the 3′ end of the second L-nucleotide strand 401, and the second copy of the active molecule 405 is attached to the second copy of the linker 404. Complementarity between the first L-nucleotide strand 103 and the second L-nucleotide strand 401 allows hybridization 500.

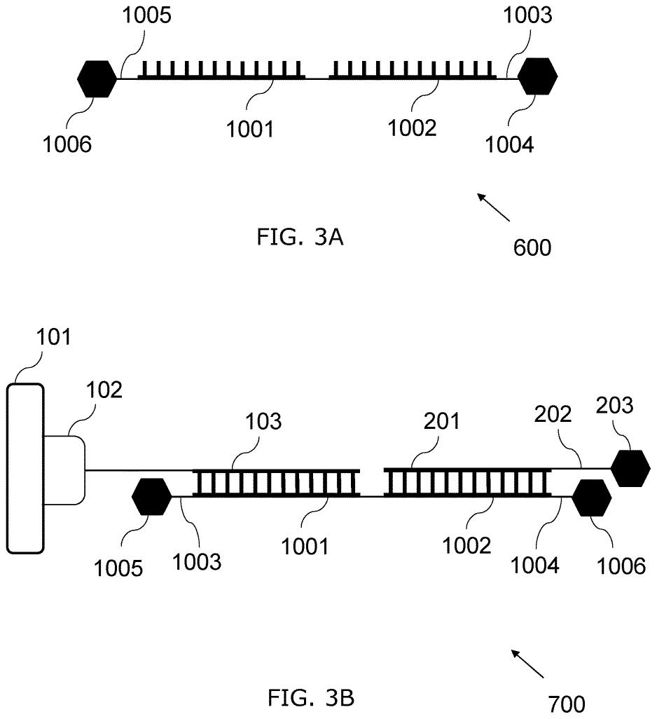

FIG. 3 shows an embodiment of the invention, where the second module 600 comprises two copies of a second L-nucleotide strand 1001, 1002, two copies of a linker 1003, 1005, and two copies of an active molecule 1004, 1006. The 5′ end of the first copy of the second L-nucleotide strand 1001 is attached to the 3′ end of the second copy of the second L-nucleotide strand 1002. The first copy of the linker 1003 is attached to the 5′ end of the second copy of the second L-nucleotide strand 1002. The first copy of the active molecule 1004 is attached to the first copy of the linker 1003. The second copy of the linker 1005 is attached to the 3′ end of the first copy of the second L-nucleotide strand 1001. The second copy of the active molecule 1006 is attached to the second copy of the linker 1005.

In an embodiment 700, one copy of the first module 100 and two copies of the second module 200, 600 hybridise. The two copies of the second module 200 and 600 are not identical. The first copy of the second module 600 hybridises to the first module 100, and the second copy of the second module 100 hybridises to the first copy of the second module 600.

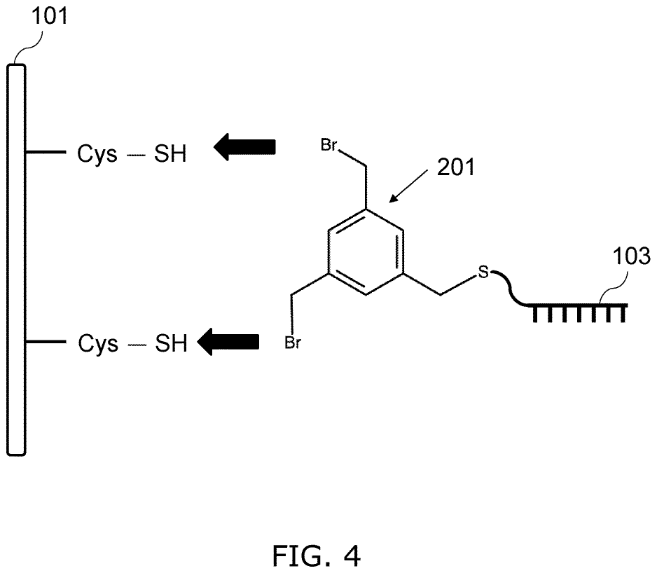

FIG. 4 shows binding of the peptide to the molecular staple with the first L-nucleotide strand attached.

The staple is conjugated to thiol groups on Cysteins present in the peptide. The staple used is 1,3,5-tris(bromomethyl)benzene (1,3,5-TBMB). The staple linker is attached to the peptide via a thiol-bromo crosslinking reaction.

1. A modular platform for molecular conjugation, comprising a first module and a second module linked together, characterized in that:
the first module comprises a peptide, a molecular staple, and a first L-nucleotide strand, and
the second module comprises a second L-nucleotide strand, at least one linker, and at least one active molecule,
wherein the second L-nucleotide strand is complementary to the first L-nucleotide strand, and
wherein the peptide and the at least one active molecule are linked together through the first and the second L-nucleotide strands hybridizing to form a double-stranded duplex.

2. The modular platform of claim 1, wherein the modular platform for molecular conjugation comprises two or more copies of the second module.

3. The modular platform of claim 1, wherein the second module comprises two active molecules.

4. The modular platform of claim 1, wherein the second module comprises two different active molecules.

5. The modular platform of claim 1, wherein the active molecule is one of: a cytotoxic agent, an immunostimulatory agent, an immunosuppressive agent, a radiosensitiser.

6. The modular platform of claim 1, wherein the active molecule is one of: a peptide, a protein, a nucleic acid, a nanobody.

7. (canceled)

8. The modular platform of claim 1, wherein the active molecule is one of: a Positron Emission Tomography (PET) tracer, a fluorescent dye, a contrast agent, a paramagnetic molecule, an ultrasound sensitizer, a radioactive isotope, an alpha-emitter, a beta emitter, a gold-based agent.

9. The modular platform of claim 8, wherein the contrast agent is gadolinium-based, iron-based, or manganese-based.

10. The modular platform of claim 1, wherein the staple contains at least one functional group enabling formation of a covalent bond with the first L-nucleotide strand.

11. The modular platform of claim 1, wherein the staple contains at least one of: a bromomethyl group, an azide group.

12. The modular platform of claim 1, wherein the staple contains at least one of:
1,3,5-tris(bromomethyl)benzene (1,3,5-TBMB),
1,2,3-tris(bromomethyl)benzene (1,2,3-TBMB),
1,2,3,5-tetra(bromomethyl)benzene (1,2,3,5-TBMB).

13. The modular platform of claim 1, wherein the hybridization of the first and the second L-nucleotide strands is reversible, allowing dynamic assembly and disassembly of the conjugation system.

14. The modular platform of claim 1, wherein the length of the first L-nucleotide strands is between 15 and 80 nucleotides.

15. The modular platform of claim 1, wherein the length of the second L-nucleotide strands is between 15 and 40 nucleotides.

16. (canceled)

17. The modular platform of claim 1, wherein the first and/or the second L-nucleotide strands are chemically modified to contain at least one thiol group.

18. (canceled)

19. (canceled)

20. The modular platform of claim 1, wherein the first and/or the second L-nucleotide strands are chemically modified to contain at least one amine group.

21. The modular platform of claim 20, wherein the amine group is located at the 5′ and/or 3′ end of the first and/or the second L-nucleotide strand.

22. A method for preparing the modular platform of claim 1 for the conjugation of a peptide with an active molecule, the method comprising:
conjugation of a staple of the peptide to a first L-nucleotide strand,
stapling of a Cysteine-containing peptide,
conjugation of the active molecule to a second L-nucleotide strand, and
hybridization of the first and the second L-nucleotide strands and formation of a double-stranded duplex.

23. The method of claim 22, wherein after thermal dissociation of the double-stranded duplex, the second module may be replaced by an alternative second module comprising a different active molecule.

24. The method of any of claim 22, wherein the hybridization between the first and second L-nucleotide strands is urea, formamide and/or acetonitrile sensitive, and dissociation of the duplex can be induced by incubation of the system in urea, formamide, and/or acetonitrile solution.

25. (canceled)
